(19) United States
(12) Patent Application Publication
(10) Pub. No.: US 2006/0037087 A1
(43) Pub. Date: Feb. 16, 2006

(54) pMvB for targeted probiotics

(76) Inventors: Marius Jacobus van Belkum, Edmonton (CA); Michael E. Stiles, Edmonton (CA)

Correspondence Address: WILLIAM J BUNDREN;THE LAW OFFICE OF WILLIAM J BUNDREN, 734 LaRue Road, Millersville, MD, 21108, US

(21) Appl. No.: 10/916,641

(22) Filed: Aug. 12, 2004

(51) Int. Cl. A01K 67/027; A61K 38/16; A61K 48/00

(52) U.S. Cl. 800/10; 514/44; 514/12

(57) ABSTRACT

The present invention is compositions and methods for treating scours comprising a vector substantially as shown in FIG. 1.

### I. FIELD OF THE INVENTION

The present invention relates to a plasmid which can be used, in particular, for transferring a heterologous gene into, and expressing it in, a bacterium, preferably a lactic acid bacterium.

### II. BACKGROUND OF THE INVENTION

Clinical cases of E. coli post-weaning diarrhea (PWD) or scours have been increasingly observed in Canada during the last several years. The receptor for the predominant serogroup associated with PWD can only be found in about 20-30% of animals. Further, available treatments for PWD or scours typically results in significant weight loss.

Notwithstanding the usefulness of the above-described methods, a need still exists for an effective treatment of PWD or scours. A need further exists for a treatment that promotes weight gain or, at a minimum results in no further weight loss. A need also exists for a treatment protocol that does not reduce the health and/or commercial value of the animal

### III. SUMMARY OF THE INVENTION

This invention provides a composition and method for treating E. coli infections, more specifically, infections that result in post weaning diarrhea or scours. The compositions and methods of the present invention involve recombinant vectors that are effective against E. coli infections, and have the added feature of promoting weight gain.

A vector of the present invention may be derived from lactic acid bacteria, in particular lactic acid bacteria of the genus Lactobacillus, in particular Lactobacillus plantarum. The plasmids according to the invention can advantageously be stably transferred into lactic acid bacteria which belong to the genera Carnobacteria, Leuconostoc; Lactobacillus, Pediococcus, and Enterococcus,

An exemplary vector of the present invention comprises a vector substantially as shown in FIG. 1.

The invention, therefore, also relates to a plasmid as previously defined, the (proper antecedent) plasmid comprising the nucleotide sequence SEQ ID No. 1 or a sequence which differs from this sequence by the insertion, deletion or mutation of from one to several base pairs, and which retains the ability to replicate.

The invention therefore also relates to a vector as shown in FIG. 1, the vector comprising the nucleotide sequence or sequences as shown, or a sequence which differs from this sequence by the insertion, deletion or mutation of one or several base pairs and which retains the ability of the plasmid to replicate stably in suitable bacterial host cells, e.g., lactic acid bacteria.

The plasmids, comprising, where appropriate, a heterologous nucleotide sequence inserted into the plasmid, are introduced into the host cells using any known technique. Exemplary techniques include but are not limited to transformation (or gene-transfer), in particular that the gene-transfer technique developed by electroporating lactic acid bacteria, in particular Carnobacteria, Leuconostoc, Lactobacillus and Pediococcus.

The invention also relates to bacterial host cells which harbor a plasmid according to the invention, in particular harboring the plasmid pMvB.

Because of the breadth of host cells that can be used for transformation purposes, the plasmids according to the invention constitute outstanding tools for cloning and expressing heterologous nucleotide sequences in host lactic acid bacteria.

In particular, the plasmids according to the invention can be used for expressing heterologous proteins, such as bacteriocins, and proteins for resistance to these bacteriocins, also termed immunity proteins, and/or a protein for resistance to an antibiotic, for example erythromycin, in host cells, in particular lactic acid bacteria.

### IV. BRIEF DESCRIPTION OF THE DRAWINGS

FIG. 1 illustrates an expression vector pMvB of the present invention.

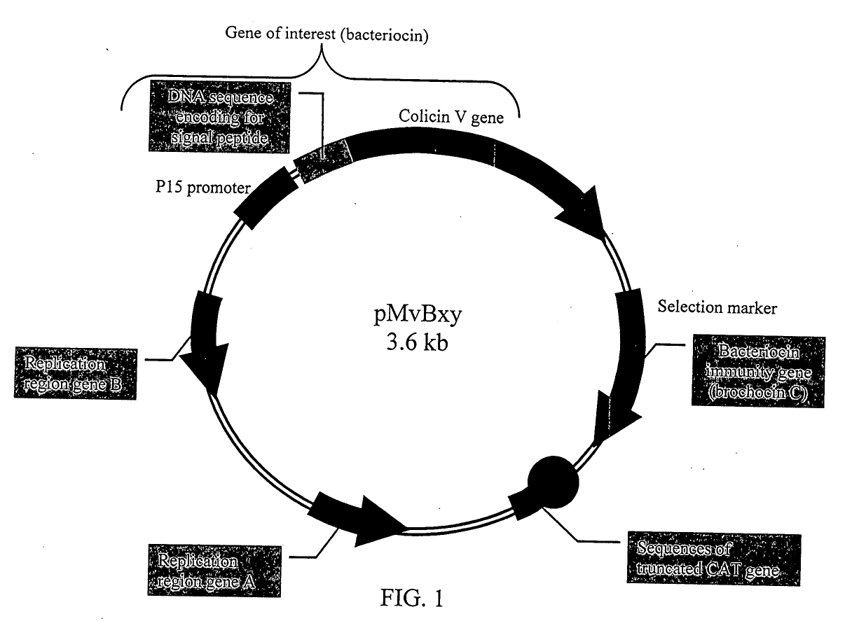

### V. DETAILED DESCRIPTION OF THE INVENTION

The present invention is expression vector pMvB, comprising a suitable promoter, e.g., p15; a signal peptide encoding DNA, e.g., divergicin A signal peptide; a gene encoding a polypeptide, e.g., encoding a bacteriocin, including but not limited to colicin V; a selection marker, including but not limited to a bacteriocin immunity gene, e.g., brochocin C; and a suitable replication region or regions, e.g., pCat (a commercially available plasmid).

Another embodiment of the present invention includes a host cell transformed by an expression vector of the present invention, including but not limited to pMvB.

Another embodiment of the present invention includes food-grade vector (pMvB and methods of use thereof.

Another embodiment of the present invention includes a vector and methods of use thereof wherein the vector includes a bacteriocin immunity gene selected from the group consisting of brochocin C, and carnobacteriocin A.

Another embodiment of the present invention includes an animal feed comprising a host bacteria transformed with an expression vector of the present invention, a bacteriocin produced by a transformed host of the present invention, or combinations thereof.

Another embodiment of the present invention includes a probiotic composition comprising a host bacteria transformed with an expression vector of the present invention, a bacteriocin produced by a transformed host of the present invention, or combinations thereof.

Another embodiment of the present invention includes a method of treating bacterial infections in animals using a composition comprising a host bacteria transformed with an expression vector of the present invention, a bacteriocin produced by a transformed host of the present invention, or combinations thereof.

In preferred embodiments of the invention, sequences from a pCAT plasmid that is not required and/or unwanted (such as antibiotic markers and mobilization genes) are deleted to result in a 2.7-kb fragment of pCAT that may be used as replicon. In accordance with the present invention, several additions are made to the pCaT replicon, including but not limited to any desired genes (such as bacteriocin and immunity genes), promoters (such as P15) and expression signals. In accordance with the present invention, a replication sequence (or replication sequences) suitable for use in a lactic acid bacteria host may be used. Suitable replication sequences include but are not limited to the replication region of pCaT. In preferred embodiments of the invention, the replication sequences include a PCAT segment derived from L. plantarum. A suitable promoter includes but is not limited to the sequences shown in Seq. ID No. 1.

In accordance with the present invention, any promoter suitable for use with expressing a bacteriocin gene may be used. Suitable promoters include but are not limited to P15. In preferred embodiments of the invention, the expression vector includes a p15 promoter, operatively associated with the bacteriocin gene of interest. In accordance with the present invention, a promoter having nucleotide sequences corresponding to Seq. ID No. 2 may be used.

In accordance with the present invention, any signal peptide suitable for use with expressing a bacteriocin gene may be used. Suitable signal peptides include but are not limited to signal peptide of divergicin A. In preferred embodiments of the invention, the expression vector includes a divergicin A signal peptide, operatively associated with the bacteriocin gene of interest. In accordance with the present invention, a signal peptide having nucleotide sequences corresponding to Seq. ID No. 3 may be used.

In accordance with the present invention, any bacteriocin gene may be used. Suitable bacteriocin genes include but are not limited to colicin V, colicin YN, leucocin A, brochocin C, In preferred embodiments of the invention, the expression vector includes a colicin V gene. Exemplary nucleotide sequences for a bacteriocin are well known to those skilled in the art. See, for example, U.S. Pat. No. 6,403,082 (Stiles, et al.)

In accordance with the present invention, any selection marker suitable for use with expressing a bacteriocin gene may be used. Suitable selection markers include but are not limited to immunity genes for carnobacteriocin A, piscicolin 126 and brochocin C. In preferred embodiments of the invention, the expression vector includes a bacteriocin immunity gene, preferably a brochocin C immunity gene, operatively associated with the bacteriocin gene of interest. Exemplary nucleotide sequences for an immunity gene are well known to those skilled in the art. See, for example, U.S. Pat. No. 6,403,082 (Stiles, et al.)

The invention also includes a host transformed with an expression vector of the present invention, and/or the bacteriocin produced by the host, may be used to treat animals, such as pigs. The treatment may include its use as a feed additive, e.g., to promote weight gain; or its use as a probiotic to beneficially affect the host by improving the properties of the indigenous microflora, e.g., in a disease or condition such as scours.

Definitions

The term gene as used herein refers to a DNA sequence, including but not limited to a DNA sequence that can be transcribed into mRNA which can be translated into polypeptide chains, transcribed into rRNA or tRNA or serve as recognition sites for enzymes and other proteins involved in DNA replication, transcription and regulation. These genes include, but are not limited to, structural genes, immunity genes and secretory (transport) genes.

The term vector as used herein refers to any DNA material capable of transferring genetic material into a bacterial host organism. The vector may be linear or circular in topology and includes but is not limited to plasmids, food grade plasmids or bacteriophages. The vector may include amplification genes, enhancers or selection markers and may or may not be integrated into the genome of the host organism. The term “secretion vector” refers to a vector designed to provide secretion of a protein from the host organism.

The term plasmid as used herein refers to a vector that is able to be genetically modified to insert one or more genes.

The term signal peptide as used herein refers to amino-terminal amino acid residues that, when attached to a target polypeptide, permits the export of the target polypeptide from the cell and cleavage of the signal peptide. The signal peptide accesses the general protein secretion pathway. An example of a signal peptide is the Divergicin A signal peptide described in U.S. Pat. No. 6,403,082, incorporated herein by reference. Other signal peptides can be used and are known to those skilled in the art.

The term “leader peptide” herein refers to an amino-terminal amino acid residues that, when attached to a target polypeptide, permits the export of the target polypeptide from the cell and cleavage of the leader peptide. The leader peptide includes but is not limited to 15-24 amino acid residues that are able to direct export of polypeptides from the cell using the dedicated transport system of the cell. The leader peptide sequence shares similarity on their primary structure and contain a conserved processing site of glycine-glycine residues or glycine-alanine residues at positions −2 and −1 of the processing site. The dedicated transport system includes but is not limited to the ATP binding cassette (ABC) transporter required for leader peptide-dependent transport. There are many different leader peptides that could be used including, but not limited to, leucocin A, colicin V, carnobacteriocin A, carnobacteriocin B2, enterocins 900 A and B or carnobacteriocin BM1.

A “processing peptide” includes both leader peptides and signal peptides, and may refer to both simultaneously, as used herein.

The term “cassette” herein refers to a DNA sequence containing a series of bacteriocin genes and if necessary their respective immunity genes, appropriate promoters, ribosomal binding site (RBS) and if necessary terminating sequences and other regulatory DNA sequences. The cassette consists of two or more nucleotide sequences encoding a structural (bacteriocin or other substrate) gene linked directly to DNA sequences encoding for an amino-terminal signal peptide compatible for export through the general export pathway of the cell or linked to the leader peptide DNA sequence compatible for export through the dedicated transport system of the cell or through a compatible dedicated transport system also inserted into a vector used to transform the cell.

The term food-grade as used herein refers to the origin of the DNA material. Food-grade indicates that a regulatory agency would consider the substance as coming from a food source and therefore suitable for inclusion in food or food products. Organisms that are food-grade, such as lactic acid bacteria and other established genera of starter organisms, can be added directly to food without concern for pathogenicity.

The term a bacteriocin as used herein refers to polypeptides and the like produced by the bacteria that inhibit one or more bacterial species. This includes, but is not limited to, polypeptides that were derived from specific strains of bacteria, proteins that were derived from other types of organisms or proteins developed through genetic engineering. The bacteriocin can be bacteriostatic or bactericidal.

The term “class II bacteriocin” herein refers to a bacteriocin which includes but is not limited to small or moderate sized polypeptides. This includes but is not limited to heat resistant polypeptides and heat sensitive polypeptides that do not undergo post-translational modification except for cleavage of the leader or signal peptide and in some cases formation of disulfide bridges. This protein must have suitable size and properties so that it can be exported from a cell. Class II bacteriocins include, without limitation, piscicolin 126, leucocin A, brochocin-C, enterocins A and B, divergicin A, carnobacteriocins A, BM1 and B2.

The term class II protein herein refers to a small protein or polypeptide which does not undergo post-translational modification except for cleavage of the leader or signal peptide and in some cases the formation of disulfide bridges. This protein must be a suitable size and physico-chemical properties so that it can be exported from a cell. Many such proteins or polypeptides are known. One of ordinary skill in the art can determine which proteins would be suitable without undue experimentation. These proteins include, but are not limited to, enzymes, hormones, inhibitors that are polypeptides or other regulatory polypeptides or proteins.

The term immunity gene as used herein refers to a gene that produces a protein that protects the host organism against the bacteriocin that it produces.

The term host organism as used herein refers to a living bacterium or microorganism capable of taking up the plasmid vector, expressing the genes and producing the desired peptide(s). If the secretion of the desired polypeptide is required, the host organism must have functional transport proteins compatible with the signal or leader peptide attached to the polypeptide to be exported or it must be able to incorporate the dedicated transport protein(s) necessary for the leader peptide-dependent export of the substrate generated from vector DNA. Host organism capable of utilizing the divergicin A signal peptide use the general secretory (sec-) pathway of the cell (for additional information see Pugsley (1993) and Simonen and Palva (1993) and references therein).

The term transport proteins as used herein refers to proteins that are in most cases incorporated into the cell membrane of the host organism and use energy in the form of adenosine triphosphate to drive the transport of protein(s) across cell membranes having a signal or leader peptide. Additional regulatory components, binding sites or enzymes may also be required for the functioning of the transport proteins.

The term homologous transport system indicates that the transport system and the leader peptide or signal peptide used to export polypeptides are derived from the same host.

The term heterologous transporter system indicates that the transport system and the leader peptide or signal peptide used to export polypeptides are derived from the different hosts. Divergicin A, for example of a signal peptide that can be used in heterologous transport systems. Homologous transporter systems can be used in homologous or heterologous bacteria if the transport system is introduced into the host organism.

The term susceptible bacterium refers to a species or strain of bacteria that is inhibited by the presence of one or more bacteriocins in its environment. Preferred susceptible bacteria are inhibited by brochocin-C, colicin V, or any other bacteriocin.

We claim:

1. A vector comprising nucleotide sequences for at least one pre-selected polypeptide; a promoter operatively associated with said nucleotide sequences; a signal peptide operatively associated with said nucleotide sequences; a selection marker comprising nucleotide sequences corresponding to a bacteriocin immunity gene; and a suitable replicon.

2. The vector of claim 1 wherein the promoter comprises p15 nucleotide sequences.

3. The vector of claim 1 wherein the pre-selected polypeptide comprises nucleotide sequences corresponding to a bacteriocin gene.

4. The vector of claim 3 wherein the polypeptide is colicin V.

5. The vector of claim 1 wherein the bacteriocin selection marker is brochocin C.

6. The vector of claim 1 wherein the signal peptide is col V.

7. A composition comprising a vector of claim 1.

8. A method of treating a E. coli infection comprising administering an effective amount of a composition comprising a vector of claim 1.


SEQUENCE LISTING (3 sequences)

SEQ ID NO: 1 - DNA, 2723 bp, artificial sequence
  replicon region of pCat
gcatgcaaagaaaagcacccttgctttttttgcctgcctaacggcgagtgattaggtggtcaagcgaacttcgcttgtgggtttgggcaacgcccaagatctttttatgtaacgaccatagggaggcgcaatgagcaaagcgaatgaagttaaatgtgagagcttgtctctcactccttttgtttgtgttgttttgttttattgaattgcgctcacaaacatagcgctacgcgcgcacctaatttattaataattctattaatacttctaaatacttagggaaaaaggaatggctttatatcaacgtttatagcactaatagaacaccaaaaagcacaccactcaacaccaaaaagcacaccactcaacaccaaaaagcacaccactcaacaccaaaaagcacaccactcaacaccaaaaaaataaaaatcactttactttagtgttaattagtaatagagtgtatttataaaataaaatgtatgttcgaaaggaagaaaatttatggtcgataaacgcaatatgttatgggtcagtgaccgtaactttaataaagaagattttaaagaccaacaatattttcaggtggctgaacataatgacctaattactaaggcacgtcatgatttaaatgcccgtgaattgaagattatggactttgttgtttctaagattaaacctgatgataaacactttaatatcattcatacgtcaatgtatgagctgtctaatgtccttgaattaaagcgtagcggtcgtacgtactcgcaacttgcccaaaatttagaagacatgcgtgctaaaagccttcgtatttatagtgagacggatcgtcgtattactctaacggggtggtttgaagttgtgcatatttgggaaaatggacaacttcaaatcaaaatcaatgaagaatttgcgccatttttgttgcaattaaaagataaagggcactacacccaatacttactcgttgataccgttaaactgaaaagtaaatattctattttgttatacaaattgatccgtgaagctgataaagataacggtagttcaattgccattgttcaaggaacgcctgatgagtggaaagaatggttaggttcgcctgaaagttacacctatggacgtttaaaagataaaatattaaacccagctattgcagaaattaaccttgaaattgatgatatggacttatctttatttcaagcacgccgtggtcgtgaggtagttcaagtcgaaattcacaataattttattcgtaacccacgtttttaatataaggggggaaatgatgttcataataaaaagagggataccgccctagcctccacagctcacggtatccctaaatttagcaaaacaaccaaccatttgaaaggaagtttcactatgaagaatagtatcacaaaaataagtctagttgtatcagttatcagtctgttggtagtgggttttcttgcgctacaaatgcatgaactcaccacaattacaacgagttcacacaaatttaacttataagttatgtcttttgaaccttgagtgagaatgtagtcgagtactaggcatgagaattttttgtaggtttcaaaaaatattacccgttcttgacggaatgaacgactgaaaaccgaagttagttacatattatatgtacgaaggcatttcatttacacaagattcttgacgctaccatatctttttggaaatgtcgccctttaaaataattttccatcatcgctacctcgctttttaggctaattatactcaaaataagtttacctgaaaaattttgccccagaaccagaaaaagtatatattgaatatgacaaagtaaaagcagatacgtgggatagacgtaatatgcgtgttgaatttaatccaaataaactcgcacatgaagaaatgctttggttaaaacaaaacatcatcgactacatggaagatgacggttttacaagattggatttagcttttgattttgaagatgatttgagcgattactacgcaatgactgacaaagcagttaaaaaaactattttttatggtcgtaatggcaagccagaaacaaaatactttggcgttcgtgatagcgatagatttattagaatttataataaaaaacaagaacgtaaagataacgctgatgttgaagttatgtctgaaaatttatggcgtgtagaaatagaattaaaaagaagtatggttgattactgggatgattgttttaatgatttacatattttaaaaccagattggaaaactatcgaacgtacttctgatagagcaatggtttttatgttgttgaatgatgaagaagaatggggaaaattagaaagacgtactaagaataaatataaaaaattaattaaagaaatatctctaattgatttaactgatttaatgaaatcgactttaaaagcgaacgaaaaacaattgcaaaagcagattgatttttggcaacgtgaatttagattttggaagtaaagtaactttgttttatacaaaatgttactttagtataattaacgtttaagaggtgacacaatatatgaaaaaatcagaggattattcctcctaaatacaaagagataagatgaaatttaggaggaaaattatatatgacttttaatattattaatttggaaacttgggatagaaaagaatattttaatcattatttcaatcaacaaacaacttacagtgttactaaagaatttgatatc

SEQ ID NO: 2 - DNA, 274 bp, artificial sequence
  p15 promoter
gatccaccaaatgtccattttttaaatcacctctttcattaaatctcattagttagtgtaccatatttgaccgtctaaaagtattttgtaaaataaatttaatatttaaatgaacaaaacttaagagttttaaatgtaggaaaatagtttaaattccttgtaaagtaaaaaaactttacaaagtataaaaaaaagctagcaacttttgcaaaaaaatgatatgatattcaagtccgaagataacaaaaatgtttcttcaaatatggttgaatgc

SEQ ID NO: 3 - DNA, 124 bp, artificial sequence
  divergicin A signal peptide
atcttggtatcacaaactaatttggaggttggtatatatgaaaaaacaaattttaaaagggttggttatagttgtttgtttatctggggcaacatttttctcaacaccacaacaagcttctgct